(19) United States
(12) Patent Application Publication
(10) Pub. No.: US 2004/0176447 A1
(43) Pub. Date: Sep. 9, 2004

(54) Method of treating an infection

(76) Inventor: Arthur Vanmoor, Boca Raton, FL (US)

Correspondence Address: OTTO S KAUDER, 5382C Venetia Ct., Boynton Beach, FL, 33437, US

(21) Appl. No.: 10/793,554

(22) Filed: Mar. 4, 2004

Related U.S. Application Data
(63) Continuation-in-part of application No. 10/382,927, filed on Mar. 6, 2003.

(51) Int. Cl. A61K 31/205; A61K 31/22

(52) U.S. Cl. 514/546; 514/556

(57) ABSTRACT

There is disclosed a method of treating an infection in a person in need of such treatment, which comprises the administration to such person of an effective amount of at least one compound represented by formula (I)

X—CH₂—CH(OY)₁₋ₚ(H)ₚCH(NH₂⁺)ₚCOO⁻ (I)

wherein X represents a trimethylammonio group (CH3)3N+ or a carbamoyl group (CONH₂), Y is hydrogen or an acetyl (CH₃CO) group, and p is zero or one, provided that when X is a carbamoyl group p is one, and when X is a trimethylammonio group p is zero.

### CROSS-REFERENCE TO RELATED APPLICATION

This application is a continuation-in-part of copending application Ser. No. 10/382927 filed 6 Mar. 2003.

### BACKGROUND OF THE INVENTION

1. Field of the Invention

This invention relates to a method of treating a person suffering from any of the large variety of known infections and associated discomfort with an agent effective in helping the affected person to subdue the infection and minimize associated pain and discomfort.

2. Description of Related Art

Infection has been defined as the invasion and multiplication of microorganisms in body tissues, especially that causing local cellular injury due to competitive metabolism, toxins, intracellular replication, or antigen-antibody response, see “Miller-Keane Encyclopedia and Dictionary of Medicine, Nursing, and Allied Health” (New York and London, W. B. Saunders Co., 1992) pages 764-766, here incorporated by reference.

As is well known, remedies for infections have been sought for generations by a great variety of methods, and with the increasing application of science some successes have been achieved, particularly in the area of sulfa drugs and antibiotics. However, bacteria, viruses, and other microorganisms believed responsible for the occurrence of infection are known to mutate rapidly, thus giving rise to new strains requiring the development of new remedies. Moreover, the search for such better remedies is enormously costly. For economic reasons, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

The human body is enormously resilient. From the detoxification of foreign ingested chemicals in the liver to the proliferation of white blood corpuscles (also called leukocytes, or pus when seen to exude from the body) that can neutralize infectious agents, the body's resources are manifold and still insufficiently understood and utilized in deliberate and targeted ways.

To date, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds are disclosed in U.S. Pat. Nos. 5,626,831, 5,707,967, 5,708,029. and 5,767,157 as effective against facial pimples, the common cold; headache; elevated blood pressure, and pain in a joint.

U.S. Pat. No. 6,479,547 discloses treating an infection with an aliphatic sulfur compound.

Cook et al U.S. Pat. No. 3,629,451 discloses treating certain bacterial infections with an omega amino acid having three to five carbon atoms between amino and carboxyl groups. Cook et al characterizes alpha amino acids as completely ineffective.

Niibe et al WO 00/37070 discloses carbocysteine 2-amino-3-(S-carboxymethyl)thiopropionic acid as the active ingredient of a drug capable of preventing infectious disease in the pre-infective step of adhesion of bacteria to the respiratory tract.

G. Tena Chemical Abstracts 1988, vol. 109, abstract 6973k disclosed preparation of a solution of amino acids and peptides from animal origin liquids under sterile conditions for use in treating viral infections.

### SUMMARY OF THE INVENTION

In accordance with this invention, there is provided a method of treating an infection in a person in need thereof, which comprises the administration to such person of an amount of at least one water-soluble aminoacid represented by formula (I) shown below, effective in mitigating the infection.

X—CH₂—CH(OY)₁₋ₚ(H)ₚCH(NH₂⁺)ₚCOO⁻ (I)

wherein X represents a trimethylammonio group (CH3)3N+ or a carbamoyl group (CONH₂), Y is hydrogen or an acetyl (CH₃CO) group, and p is zero or one, provided that when X is a carbamoyl group p isone, and when X is a trimethylammonio group p is zero.

Without intending to be bound by any theory, the present invention is believed to be beneficial in augmenting the person's innate ability to eliminate toxins from the body and to resist the initiation of the process that makes a person susceptible to infection as well as to slow down, arrest, and even reverse that process. As a result, the incidence of infection and the associated discomfort is diminished, and the quality of life is improved.

In mitigating an infection, mega-nutrient doses of 2 to 20 grams of at least one water-soluble aminoacid represented by formula (I) can be administered from one to five times daily, for a total of 2 to 100 grams per day, until monitoring shows sufficient improvement in the user's condition to permit reduction in dose level and ultimately cessation of the treatment. Daily doses of a water-soluble aminoacid represented by formula (I) in the range from 2 to 40 grams per day are preferred. Such doses can be administered in any convenient manner, as by oral administration in any of the usual dosage forms, such as tablets, capsules, solutions, and dispersions in liquid foods such as soups and fruit juices. Alternatively, there can be given sterile solutions of a water-soluble aminoacid represented by formula (I) by direct injection into the bloodstream of the person to be treated, as well as by rectal suppositories containing a water-soluble aminoacid represented by formula (I).

Infections that can be treated by the method of this invention can be located on the outside of the body or affect internal organs, and include infections caused by intracellular infectious agents and extracellular infectious agents, such as tubercle bacilli, gram-positive bacteria and gram-negative bacteria; tropical parasites including ameoba; or viruses, in particular all forms of dysentery, hepatitis, herpes, pneumonia, sexually transmitted diseases such as syphilis, and typhoid fever.

In formula (I), when X is a carbamoyl group the compound is that represented by formula II

H2NCO—CH₂—CH₂₋CH(NH₂⁺)COO⁻ (II)

When X is a trimethylammonio group the compound can be that represented by formula (III)

(CH3)3N+-CH₂—CH(OH)CH₂COO⁻ (III)

as well as its acetylated derivative represented by formula (IV)

(CH3)3N+-CH₂—CH(OCOCH3)CH₂COO⁻ (IV)

### EXAMPLE 1

A 42 year old male neglected a cut on his leg such that gangrene developed. He took 5 grams of a composition of compounds of formula I four times daily for 16 weeks and observed arrest of the gangrene after two weeks and cure after four weeks.

What is claimed is:

1. A method of treating an infection in a person in need of such treatment, which comprises the administration to such person of an effective amount of at least one water-soluble amino acid represented by formula (I)
X—CH₂—CH(OY)₁₋ₚ(H)ₚCH(NH₂⁺)ₚCOO⁻ (I)
wherein X represents a trimethylammonio group (CH3)3N+ or a carbamoyl group (CONH₂), Y is hydrogen or an acetyl (CH₃CO) group, and p is zero or one, provided that when X is a carbamoyl group p is one, and when X is a trimethylanmutonio group p is zero.

2. The method of claim 1, wherein the compound is administered orally with food.

3. The method of claim 1, wherein the compound is administered by injection into the bloodstream.

4. The method of claim 1, wherein the compound is administered by rectal suppository.

5. The method of claim 1, wherein the effective amount of the compound is administered in one to five daily doses, each dose being in the range of 2 to 20 gram.

6. The method of claim 1, wherein the total of said effective amount of the compound administered daily is in the range of 2 to 100 grams.

7. The method of claim 6, wherein the total of said effective amount of the compound administered daily is in the range of 20 to 50 grams.

8. The method of claim 1, wherein the infection is located on the outside of the body.

9. The method of claim 1 wherein the infection affects an internal organ.

10. The method of claim 1 wherein the compound is represented by formula (II)
H2NCO—CH₂—CH₂₋CH(NH₂⁺)COO⁻ (II)

11. The method of claim 1 wherein the compound is represented by formula (III)
(CH3)3N+-CH₂—CH(OH)CH₂COO⁻ (III)

12. The method of claim 1 wherein the compound is represented by formula (IV)
(CH3)3N+-CH₂—CH(OCOCH3)CH₂COO⁻ (IV)

13. The method of claim 1, wherein a plurality of compounds represented by formula (I) is administered.

14. The method of claim 1 wherein the infection is a bacterial infection.

15. The method of claim 1, wherein the infection is a virus infection.

16. The nethod of claim 1 wherein the infection is an infection by tropical parasite.

17. The method of claim 1, wherein the infection is selected from the group consisting of. dysentery, gangrene, hepatitis, herpes, pneumonia, syphilis, and typhoid fever.
